(19) United States
(12) Patent Application Publication
(10) Pub. No.: US 2006/0275867 A1
(43) Pub. Date: Dec. 7, 2006

(54) Process

(76) Inventors: Veronique Chotteau, Stockholm (SE); Caroline Wahlgren, Stockholm (SE); Yun Jiang, Stockholm (SE); Erik L. Svensson, Lidkoping (SE)

Correspondence Address: FISH & RICHARDSON PC, P.O. BOX 1022, MINNEAPOLIS, MN, 55440-1022, US

(21) Appl. No.: 11/409,288

(22) Filed: Apr. 21, 2006

Related U.S. Application Data
(60) Provisional application No. 60/728,864, filed on Oct. 21, 2005.

(30) Foreign Application Priority Data
Jun. 3, 2005 (SE) 0501299-2

(51) Int. Cl. C12P 21/06; C12N 5/06; C12N 5/08

(52) U.S. Cl. 435/69.1; 435/358; 435/366

(57) ABSTRACT

A process for cultivating animal cells producing complex proteins, wherein one plant-derived peptone or a combination of plant-derived peptones is fed to the cell culture, as well as a method for reducing the toxic effect of over-feeding amino acids during a fed-batch process for cultivating animal cells producing complex proteins.

### CROSS REFERENCE TO RELATED APPLICATIONS

This application claims priority from Swedish Patent Application No. 0501299-2, filed Jun. 3, 2005, and U.S. Provisional Patent Application No. 60/728,864, filed Oct. 21, 2005. The prior applications are incorporated herein by reference in their entirety.

### TECHNICAL FIELD

The present invention relates to a fed-batch process for cultivating mammalian cells producing complex proteins.

### BACKGROUND

The cultivation of established mammalian cell lines is currently used in the biopharmaceutical industry to produce complex proteins, e.g., glycoproteins. In particular, the use of Chinese Hamster Ovary (CHO) cells for the production of monoclonal antibodies has become more and more used. The cultivation can be performed in batch, fed-batch or perfusion modes. Other cell lines like the mouse myeloma (NSO), baby hamster kidney (BHK), human embryonic kidney (HEK-293) and human-retina-derived (PER.C6) cells are alternatives. All these cell lines have been optimized to grow in suspension cultures and are easy to scale-up using stirred tank bioreactors [Butler M. Appl. Microbiol. Biotechnol., 68: 283-291 (2005)].

A fed-batch process is a cultivation that is initiated with the cells inoculated in a cultivation basal medium or basal medium. This medium provides energy sources, amino acids, an iron source, vitamins, organic compounds, growth factors, trace elements, mineral salts, pH buffering capacity and correct osmolarity. After the cell inoculation, the feed of one or several components is started according to rules established by the operators concerning which components are fed and at which frequency and concentration. Bibila et al. [Bibila T A et al. Biotechnology Progress, 10: p. 87-96 (1994); Bibila T A, et al. Biotechnology Progress, January-February; 11(1):1-13 (1995)] described an approach for fed-batch, which was exploited in the examples of the present invention in combination with other concepts.

The feeding of nutrients in fed-batch cultivations is the main reason why the viable cell number and viability often are much higher than in a batch culture. To achieve maximal productivity the aim is to keep the viability as high as possible for as long time as possible. However, the accumulation of by-products like lactate and ammonia eventually cause the viable cell number and viability to decrease. Lactate accumulation can decrease the culture pH, and if it is desired to control pH control addition of alkali might be necessary, which causes the osmolarity in the culture medium to increase. Ammonia can permeate the cell and alter the intracellular pH. Therefore it is important to reduce the accumulation of these metabolic by-products [Gambhir A, et al. Journal of Bioscience and Bioengineering, 87(6): 805-810 (1999)]. The sensitivities to lactate and ammonia are however cell-line specific and may vary greatly between cell-lines [Lao M-S. et al. Biotechnol. Prog. 13: 688-691 (1997)].

For mammalian cells to grow, the essential amino acids need to be supplemented to the medium. It is critical to obtain a balanced supplementation of the essential and other amino acids in order to prevent possible toxic effects of overfeeding amino acids [Ducommun P, et al. Cytotechnology, 37: 65-73 (2001)].

Serum contains several growth-promoting compounds like growth factors, nutrients and hormones, and has been widely used as a supplement in media for mammalian cell cultivations. However, there are a number of disadvantages with the use of serum. Serum shows a variation in shelf-life and composition from batch to batch which requires extensive quality controls to be able to achieve reproducibility between batches. It also presents difficulties in the purification of the protein product and is often associated with high costs. The most important disadvantage with the use of animal-derived serum is however the risk of viral, mycoplasma or prion contamination, which may present a contagious risk to the biopharmaceutical product [Freshney I R. Culture of Animal cells—A manual of basic technique, Wiley-Liss, , 4^{th} ed. (2000)].

Because of the numerous functions serum has in culture media, substitutes for all growth-promoting components in serum have to be found. For example, the iron-carrier transferrin can be replaced by inorganic salts and chelating agents. The surfactant Pluronic F68 substitute serum in protecting the cells against shear stress [Burteau C C et al. In Vitro Cell Dev. Biol-Animal, 39:291-296 (2003)]. Likewise, ethanolamine and sodium selenite are considered important supplements to promote cell growth in serum-free media [Hewlett G. Cytotechnology, 5: 3-14 (1991)].

To successfully replace all important components in serum by chemically defined substitutes has however shown to be difficult. Growth requirements may vary widely between cell-lines and even between clones [Butler M. Appl. Microbiol. Biotechnol., 68: 283-291 (2005)]. Metabolic analyses may help to find important media supplementations. Microarray analysis of receptors expressed by the cells during growth can be used to identify their corresponding ligands, which can be supplemented in the media [Butler M. Appl. Microbiol. Biotechnol. 68: 283-291 (2005)].

Peptones or protein hydrolysates are cocktails of amino acids and amino acids polypeptides obtained by either enzymatic digestion or acidic digestion of proteins of a given origin, i.e. meat, yeast, lacto-albumin, soy, cotton seed, rice, wheat, etc. They have been used to help the fermentation of microorganisms, e.g. E. coli. However these results cannot be applied to animal cell cultivation since microorganisms and animal cells have very different requirements. For example, microorganisms have a less complex metabolism than animal cells and they also have the ability to synthesize amino acids that animal cells are not able to synthesize. Therefore, animal cells need a more complex media containing various nutrients like vitamins, minerals, salts, amino acids, and growth factors for being able to grow.

The supplementation of peptones for animal cell cultivation has been studied since several decades. Meat-derived peptones were one of the first peptones studied for animal cell cultivation. The elimination of serum from the cell cultivation has been facilitated by its replacement by meat derived peptones so that the same performances of cell growth and productivity could be hoped [U.S. Pat. No. 6,087,126 to Horwitz A et al.; U.S. Pat. No. 5,705,364 to Etcheverry T et al.; U.S. Pat. No. 5,691,202 to Wan N C]. It has been found that meat derived peptones can replace the need of single amino acids and that peptides can be taken up by the cells by different mechanisms than the single amino acids. To use the peptones as a supply of amino acids and in particular of glutamine in an alternative way as by single amino acid addition was also described for a series of protein hydrolysates (milk, meat, soy, wheat, rice or maize proteins) in Blom W R et al. [U.S. Pat. No. 5,741,705]. The use of peptones derived from animal source could imply a risk for contamination by viruses, mycoplasma or prions. The replacement of meat-derived peptones by plant-derived peptones from rice or soy or yeast-derived peptones in animal cell cultivation medium has been described by Keen M J et al [U.S. Pat. No. 5,633,162] and Price et al [U.S. Pat. No. 6,103,529]. Jayme D W et al., [Cytotechnology 33:27-36 (2000)] presented cell growth results where human albumin was replaced by rice, wheat and soy peptones in a VERO cell bioassay system. Some have described that peptones have other properties like stimulating the growth or anti-apoptotic in CHO batch cultivation [Burteau C C et al., In Vitro Cell Dev. Biol-Animal 39:291-296 2003]. Shlaeger E J [J. Immunol. Methods 194:191-199 (1996)] observed an improvement of the maximum cell density and viability in batch cultivation of mouse hybridomas and myeloma cells, attributed to an anti-apoptotic effect. Franek F et al [Biotechnology Progress 16 (5), 688-692 (2000)] showed that a size separated fraction of wheat flour peptone enhanced the cell growth and the productivity. Others have not observed this effect and have found that the only function was to replace the amino acids supply with a cocktail of peptones, i.e. peptones from wheat and soy of two different origins, in a Baby Hamster Kidney cell line continuous cultivation [Heideman R et al., Cytotechnology 32 (2), 157-167 (2000)].

The use of meat-derived peptone feeding in CHO cell fed-batch process has been reported by Gu X et al. [Biotechnology and Bioengineering, 56:353-360 (1997)], where they have observed that this peptone feeding was equivalent to feeding single amino acids but did not bring improvement to the cell growth, the cell viability or the productivity.

As presented herein, other publications have described peptone feeding solely as an alternative way to feed single amino acids, i.e. no supplementary beneficial effect on the cell growth, cell viability or the productivity has been observed with peptone feeding. An effect of enhancement in the cell growth and/or cell viability has been described for batch cultivation where the peptone is present from the beginning of the cultivation and is not fed. The present invention provides a supplementary beneficial effect on the cell growth and/or the cell viability, accompanied by an enhancement of the productivity, by feeding a peptone or a combination of peptones.

Further, the present invention aims at a process for cultivating animal cells wherein the use of peptones derived from animal source is excluded for the sake of the patient safety. Peptones derived from a plant source reduce the risk of contamination by viruses, mycoplasma or prions.

### BRIEF DESCRIPTION OF THE DRAWINGS

FIG. 1 shows the titre improvement obtained with the fed-batch strategy compared to the batch.

FIG. 2 presents the viable cell density and the cell viability increase after peptone addition in spinner 3 in comparison with no peptone addition and a batch control.

FIG. 3 compares the product titres in different spinners, wherein peptones are added in spinner 3, and a batch control.

FIG. 4 presents an increase in viable cell density and viability when peptones were added to spinner 6 when the viable cell density and the cell viability have begun to decrease.

FIG. 5 shows further accumulation of antibody in spinner 6 when peptones were added when the viable cell density and the cell viability had begun to decrease.

FIG. 6 compares the cell specific productivity in different spinners, wherein peptones were added in spinner 6 after the viable cell density and the viability had begun to decrease.

FIG. 7 shows an improvement of the cell viability and hence the process longevity when a combination of cotton seed and pea protein hydrolysates was fed to the culture in a 3 L bioreactor.

FIG. 8 shows an increase of antibody production when a combination of cotton seed and pea protein hydrolysates was fed to the culture in a 3 L bioreactor.

FIG. 9 shows that feeding cotton seed and pea protein hydrolysates increased the viable cell number and cell viability in a fed-batch culture using a disclosed serum-free medium.

FIG. 10 shows that feeding cotton seed and pea protein hydrolysates increased the antibody production in a fed-batch culture using a disclosed serum-free medium.

FIG. 11 illustrates that the viable cell number was increased in fed-batch cultures fed with the cotton seed and pea protein hydrolysates as compared to the batch culture and that the toxic effect of the amino acid cocktail feeding was partially neutralized.

FIG. 12 illustrates that the cell viability was increased in fed-batch cultures fed with the cotton seed and pea protein hydrolysates as compared to the batch culture and that the toxic effect of the amino acid cocktail feeding was partially neutralized.

FIG. 13 illustrates that the antibody production was increased in fed-batch cultures fed with the cotton seed and pea protein hydrolysates as compared to the batch culture and that the toxic effect of the amino acid cocktail feeding was partially neutralized.

FIG. 14 shows increased viable cell number and cell viability when a combination of cotton seed and pea protein hydrolysates was fed to the culture and that the addition of the peptones partially neutralized the toxic effect from over-feeding of the amino acids.

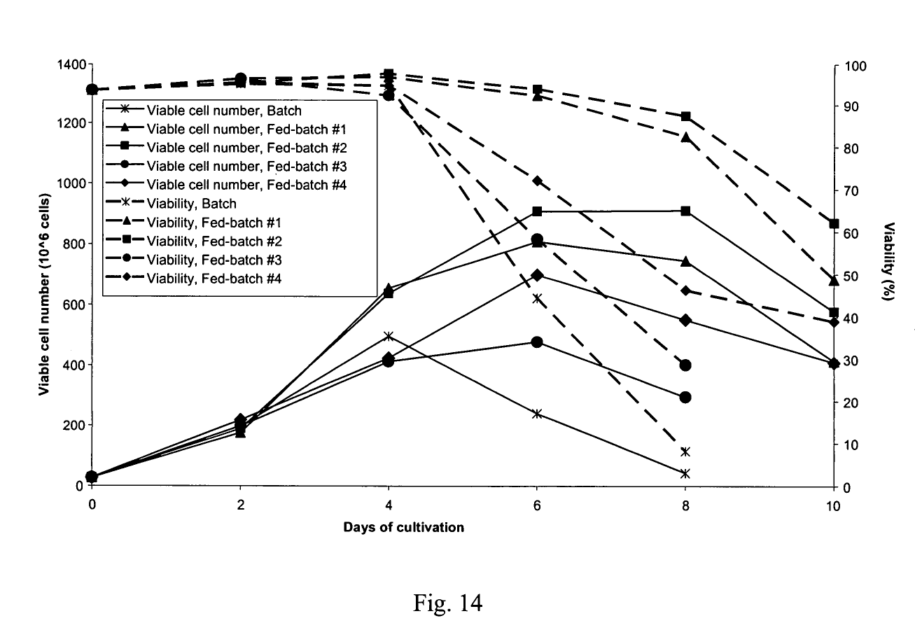

FIG. 15 shows an increase of antibody production when a combination of cotton seed and pea protein hydrolysates was fed to the culture and that the addition of the peptones partially neutralized the toxic effect from over-feeding of the amino acids.

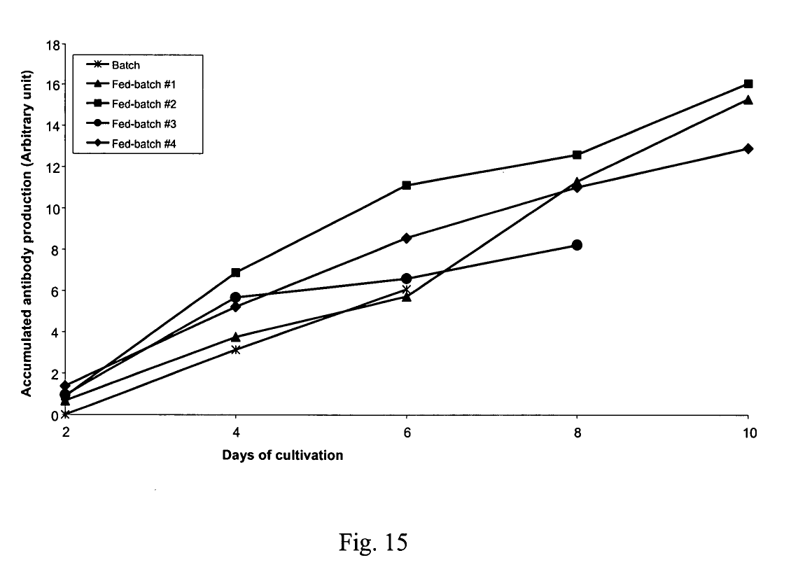

### DETAILED DISCLOSURE

Surprisingly, it has been found that when a cocktail of selected plant-derived peptones is fed in a fed-batch process of an animal cell line, the cell growth and/or the cell viability are improved. This effect on the cells is accompanied by a productivity enhancement. Consequently, the present invention relates to a fed-batch process for cultivating animal cells, including human cells, wherein one or a combination of peptones is fed to the cell culture.

In one embodiment, the process according to the present invention is a fed-batch process, wherein one or a combination of peptones is fed to the cell culture. Specifically, the invention relates to a fed-batch process, wherein a basal medium is used for the cell inoculation and a feed medium is fed to the cell culture. The fed-batch used in the present invention comprises feeding glucose, glutamine, amino acids and concentrated feed medium. The concentrated feed medium comprises the basal medium enriched in vitamins, metals and biosynthesis precursors. The feed medium can be fed continuously, intermittently or boost-wise. In another embodiment, the present invention relates to a fed perfusion process, wherein one or a combination of peptones is fed to the cell culture.

Further, the invention relates to a process wherein the cultivated cells are secreting proteins. Preferably, the invention relates to a process wherein the cultivated cells are secreting complex proteins, such as proteins that require post-translational modifications, including glycosylation and/or phosphorylation. More preferably, the secreted proteins are antibodies.

Consequently, the present invention relates to process for cultivating animal cells, including human cells, characterized in that one peptone or a combination of peptones are fed to the cell culture in order to impede partially or completely the decrease of the viable cell density and the cell viability. Specifically, the present invention relates to process for cultivating animal cells, wherein a basal medium is used for the cell inoculation and a feed medium is fed to the cell culture, and characterized in that the feed medium contains one peptone or a combination of peptones are fed to the cell culture in order to decrease the viable cell density and the cell viability decline.

The improved cell growth and/or cell viability in the process are due to the addition of the peptone cocktail during the progressed cultivation. The peptone or combination of peptones is fed continuously, intermittently or boost-wise to the cell culture. When the same combination of peptones is present in the basal medium, i.e. from the beginning of the cultivation, it does not have the same effect.

Preferably, feeding of the peptone or combination of peptones is started at any time between cell inoculation and before the cell viability decreases below the viability at the cell inoculation. More preferably, feeding of the peptone or combination of peptones is started at any time between cell inoculation and three days before the cell viability decreases below the viability at the cell inoculation. Even more preferably, feeding of the peptones is started before the cell viability declines. Also, feeding of the peptones can be started before the cell culture reaches the stationary phase or when the cell culture has reached the stationary phase.

Surprisingly, it has been found that addition of the peptone or combination of peptones also when the viable cell density and the cell viability has begun to decrease has a beneficial effect and lead to an increase in cell density and cell viability. Therefore, the present invention further relates to a process for cultivating animal cells, wherein the peptones are added when the viable cell density and /or the cell viability has begun to decrease.

Further, it has been found that addition of the peptone or combination of peptones has a beneficial effect when the amino acid feeding is under-optimized for a fed-batch process. More specifically, the addition of the peptone or combination of peptones can partially neutralize the toxic effect from over-feeding of the amino acids during a fed-batch process, resulting in improvements of cell density, cell viability, process longevity, and productivity.

Preferably, the present invention relates to a process for cultivating animal cells, wherein peptone or combinations of peptones that are derived from plants are fed to the cell culture.

Preferably, the invention relates to a process for cultivating animal cells, wherein at least one peptone is fed to the process. More preferably, this peptone is derived from Fabaceae vicieae protein. Specifically, the peptone is derived from Pisum sativum (i.e. pea).

Alternatively, the invention relates to a process for cultivating animal cells, wherein a combination of peptones is fed to the cell culture. Preferably, the combination of peptones includes at least a peptone produced by enzymatic digest and which is derived from protein of the Fabaceae family vicieae tribe, e.g. Pisum sativum or pea. Specifically, the combination of peptones further includes peptones derived from Fabaceae glycine max protein (soy), Malvaceae seed, e.g. Malvaceae gossypium (cotton seed protein), or both.

The peptone cocktail feeding is added every day, every second day or boost-wise, during the fed-batch process corresponding to a total dose of a total concentration of 0.01 gram per litre of the cultivation volume at inoculation to 15 gram per litre of the cultivation volume at inoculation, preferably 0.01 to 11 gram per litre of cultivation volume at inoculation, more preferably, 0.01 to 5 gram per litre of cultivation volume at inoculation. The total concentration is defined as the summation of the concentrations of the individual peptones of the cocktail; the individual concentrations being in gram per litre of cultivation volume at inoculation. In the fed-batch process, the basal medium can contain or not contain peptones. If it contains peptones, these can be of the same nature or not as the fed peptone cocktail.

Preferably, the animal cells used in the process according to the present invention, are mammalian cells. More preferably, the animal cells are rodent cells. Further preferably, the animal cells are hamster cells. Even more preferably, the animal cells are CHO cells.

Definitions

“Complex protein” as used herein refers to proteins that require post-translational modifications including glycosylation and/or phosphorylation. The post-translational modifications may be important for the physical and chemical properties, folding, conformation distribution, stability, activity, and consequently, function of the proteins.

“Peptone” as used herein, is the general name of a group of heat stable, acid or enzymatic hydrolysates of proteins with animal, vegetable or yeast origin.

“Batch process” as used herein, is a process where the cultivation volume is constant and all substrate components are present from the beginning.

“Fed-batch process” as used herein, is a process where the cultivation is started by inoculating the cells in the cultivation basal medium or basal medium and where additions of various additives are performed during the cultivation.

“Perfusion” as used herein is a cultivation process in which cell clarified supernatant is removed continuously or intermittently from the cultivation bioreactor and fresh basal cultivation medium is added continuously or intermittently to the bioreactor cultivation with or without recycling part of the clarified supernatant.

“Fed perfusion” as used herein is a perfusion process where one or several components are fed in addition to the components already present in the basal medium. The supplementary fed components may be not present in the basal medium or may be present in the basal medium at a different concentration than the fed concentration.

“Cultivation basal medium” or “basal medium” as used herein, is the cultivation medium used initially for the cell inoculation of the cultivation. This medium is able to sustain animal cell growth and contains water, energy sources, amino acids, iron source, vitamins, organic compounds, mineral salts, trace elements, mineral salts, pH buffering capacity and correct osmolarity. Optionally it also contains one or several growth promoting factor(s).

“Feed medium” as used herein, is a water based mixture containing one component or more and which is fed continuously, intermittently or boost-wise to the cell culture during the fed-batch process.

A “cocktail of peptones” as used herein, is a combination of one, two, three or four peptones.

“Total cell density” as used herein, includes all the cells, i.e. both the viable cells and the dead cells.

“Cell viability” as used herein, is defined as the ratio of the viable cell density over the total cell density.

“Stationary phase” as used herein, is defined as when the cell growth has stopped and the number of cells remains constant and new cells are produced at the same rate as older cells die.

“Total dose” as used herein, is defined as the sum of all individual doses fed to the process.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention will now be further illustrated through the description of examples of its practice. The examples are not intended as limiting in any way of the scope of the invention.

### EXAMPLES

### Example 1

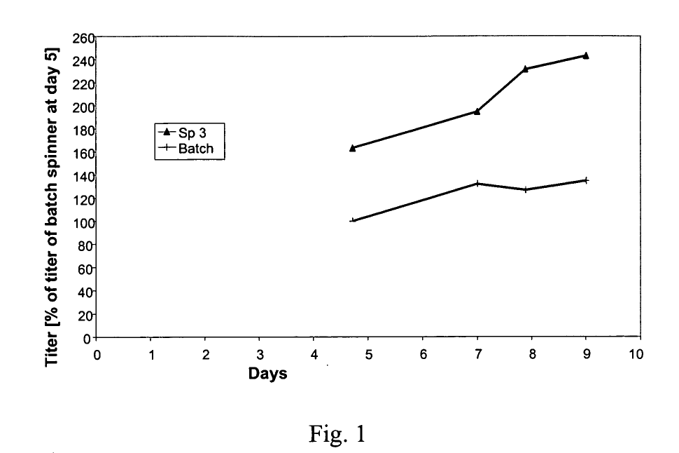

A fed-batch cultivation was performed in spinner flask with an antibody producing CHO cell line. The fed-batch cultivation was fed with glucose, glutamine, amino acid cocktail, three peptones (i.e. D, E and G) and concentrated feed medium consisting of the basal medium enriched in vitamins, metals and biosynthesis precursors. The basal medium was based on DMEM/F12 medium enriched in amino acids, surfactant, vitamins, and organic compounds. Peptone D 5 g/L had been added to this basal medium. This fed-batch strategy resulted in significant higher titre than obtained in the control batch cultivation with the same basal medium supplemented with 5 g/L peptone D. FIG. 1 shows that a significant titre improvement was obtained when a fed-batch strategy with feeding of soy, cotton seed and pea protein hydrolysates was applied (Sp 3) compared to the batch cultivation (Batch); the basal medium including soy peptone in both cultivation runs.
peptone D=soy protein hydrolysate
peptone E=cotton seed protein hydrolysate
peptone G=pea protein hydrolysate

### Example 2

A fed-batch cultivation, spinner 3, was performed in spinner flask with an antibody producing CHO cell line. The fed-batch cultivation was fed with glucose, glutamine, amino acid cocktail and concentrated feed medium consisting of the basal medium enriched in vitamins, metals and biosynthesis precursors. At day 7, after the viable cell density and the cell viability had begun to decrease, the amino acid cocktail was replaced by feeding a combination of peptones G and E (50/50%/%). The basal medium was based on DMEM/F12 medium enriched in amino acids, surfactant, vitamins, and organic compounds. Peptone D 2.5 g/L and peptone G 2.5 g/L had been added to this basal medium. As control, a parallel fed-batch cultivation, spinner 1, was performed in exactly the same conditions except that the amino acid cocktail was not replaced at day 7 by a peptone feeding but was continued in the same way as applied before in the fed-batch. A third fed-batch cultivation, spinner 2, was also performed in parallel and had exactly the same conditions as the control spinner 1 except that the basal medium had been supplemented with peptone E 2.5 g/L and peptone G 2.5 g/L instead of peptone D 2.5 g/L and peptone G 2.5 g/L. A batch cultivation was also performed in parallel using the same basal medium and supplemented with peptone D 5 g/L. Spinner 3 where a peptone G and E feeding was applied from day 7 resulted in a surprising cell increase and viability increase the day after. In the control spinner 1, the viable cell density and cell viability continued to decrease. Spinner 2 had a basal medium including the precise peptone combination G and E (50/50%/%). It can be seen from the results of viable cell density and cell viability that it was the feeding of peptones G and E in spinner 3 after day 7, which caused the viable cell density and cell viability increases and not just only the presence of the peptones G and E in the basal medium (spinner 2), i.e. during the whole cultivation.

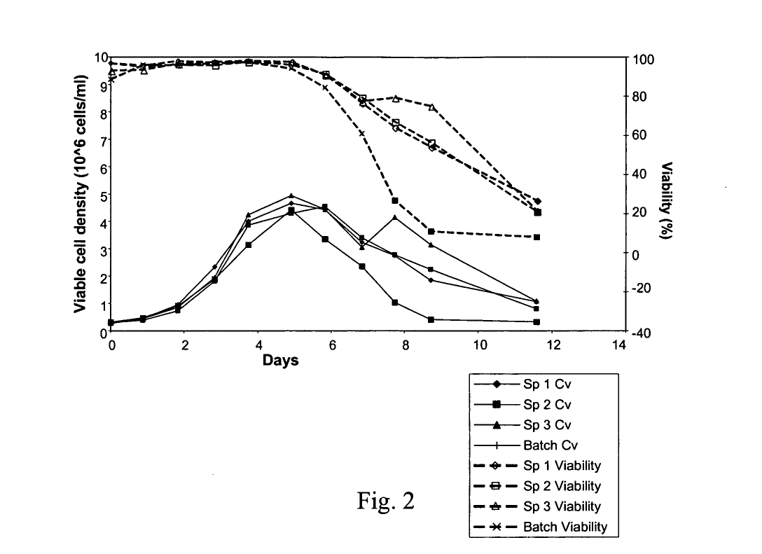

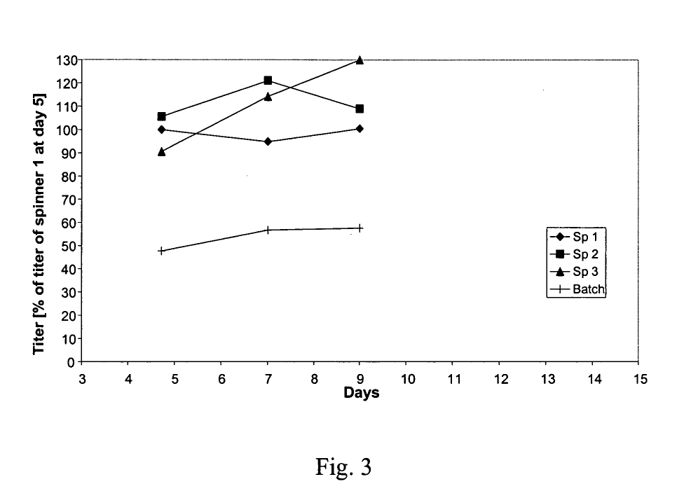

Finally the titres and the cell specific productivity results show that this benefit of feeding peptones G and E in spinner 3 resulted also in a benefit for the productivity and the cell specific productivity after day 7. FIG. 2 shows that feeding a combination of peptone E, and peptone G at day 7 resulted in an increase in cell density and cell viability in spinner 3 (Sp 3) in comparison with the fed-batch spinner 1 (Sp 1) performed in the same conditions except for the absence of the peptone feeding. FIG. 2 shows also that it was the feeding of cotton seed and pea peptones in spinner 3 (Sp 3) which gave the cell density increase since the fed-batch spinner 2 (Sp 2), which had cotton seed and pea peptones in the basal medium had a cell density and viability, which were not higher than the ones of spinner 3. FIG. 3 shows that by feeding a combination of cotton seed and pea peptones at day 7 a higher productivity was obtained after day 7 in spinner 3 (Sp 3) in comparison with the fed-batch spinner 1 (Sp 1) performed in the same conditions except for the absence of peptone feeding. FIG. 3 shows also that it was the feeding of cotton seed and pea peptones in spinner 3 (Sp 3) which gave the productivity increase since the fed-batch spinner 2 (Sp 2), which had cotton seed and pea peptones in the basal medium had a productivity, which was lower than the one of spinner 3 after day 7.
peptone D=soy protein hydrolysate
peptone E=cotton seed protein hydrolysate
peptone G=pea protein hydrolysate

### Example 3

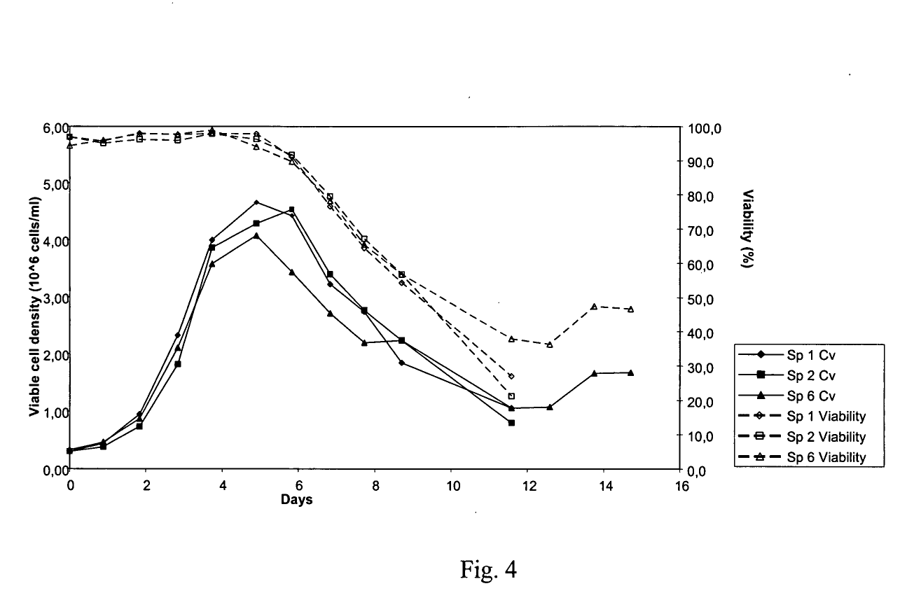

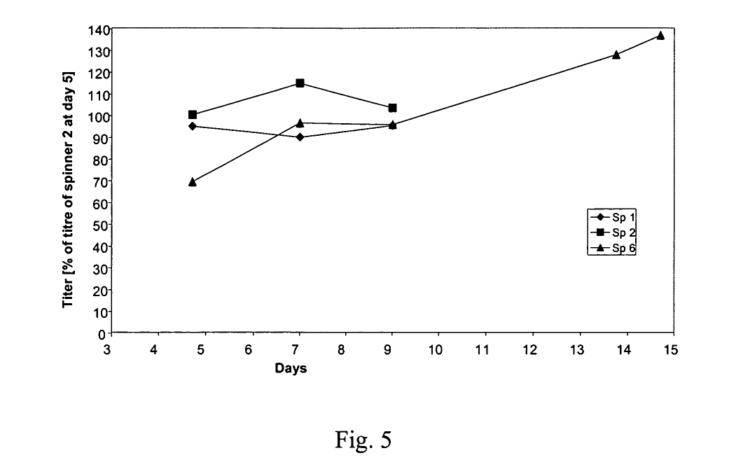

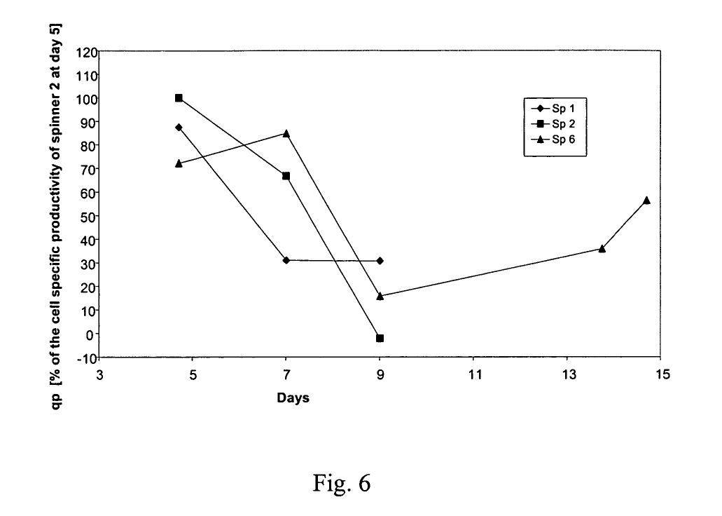

A fed-batch cultivation, spinner 6, was performed in spinner flask with an antibody producing CHO cell line. The fed-batch cultivation was fed with glucose, glutamine, amino acid cocktail and concentrated feed medium consisting of the basal medium enriched in vitamins, metals, biosynthesis precursors and pyruvate. At day 9, after the viable cell density and the cell viability had begun to decrease since four days, the viability was then 57%, which is very low, the amino acid cocktail was replaced by feeding a combination of peptones G and E (50/50%/%). The basal medium was based on DMEM/F12 medium enriched in amino acids, surfactant, vitamins, and organic compounds. Peptone D 5 g/L had been added to this basal medium. In comparison, two parallel fed-batch cultivation, spinners 1 and 2, were performed in exactly the same conditions with the following exceptions: the amino acid cocktail was not replaced at day 9 by a peptone feeding but was continued in the same way as the day before and the basal medium was supplemented with peptones D and G and with peptones G and E, respectively, and spinners 1 and 2 feed medium had not been enriched in pyruvate. Notice that the pyruvate enriched feeding in spinner 6 had not resulted in better cell density or viability than in spinners 1 and 2 and cannot be not responsible for the improvement observed after day 9 in spinner 6. It was observed that the viable cell number and the cell viability were increased in a comparable effect as observed in Example 2, confirming the results of Example 2. Notice that the cells continued to produce antibodies and that their cell specific productivity, which had decreased to 16%, increased de novo to 36% and 56% where 100% is the cell specific productivity of spinner 2 at day 5. FIG. 4 shows that feeding peptone E and peptone G at day 9 in spinner 6 (Sp 6) resulted in an increase in cell density and viability after day 9 although the cell viability at day 9 was very low, 57%. FIG. 5 shows that feeding peptone E and peptone G at day 9 in spinner 6 (Sp 6) resulted in further accumulation of antibody. FIG. 6 shows that the cell specific productivity was increased by feeding peptone E and peptone G at day 9 in spinner 6 (Sp 6).
peptone D=soy protein hydrolysate
peptone E=cotton seed protein hydrolysate
peptone G=pea protein hydrolysate

### Example 4

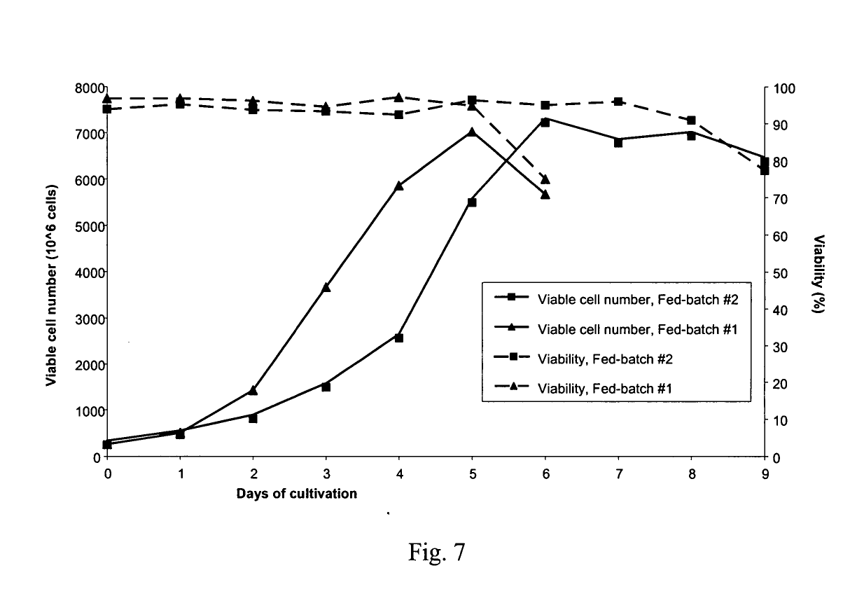

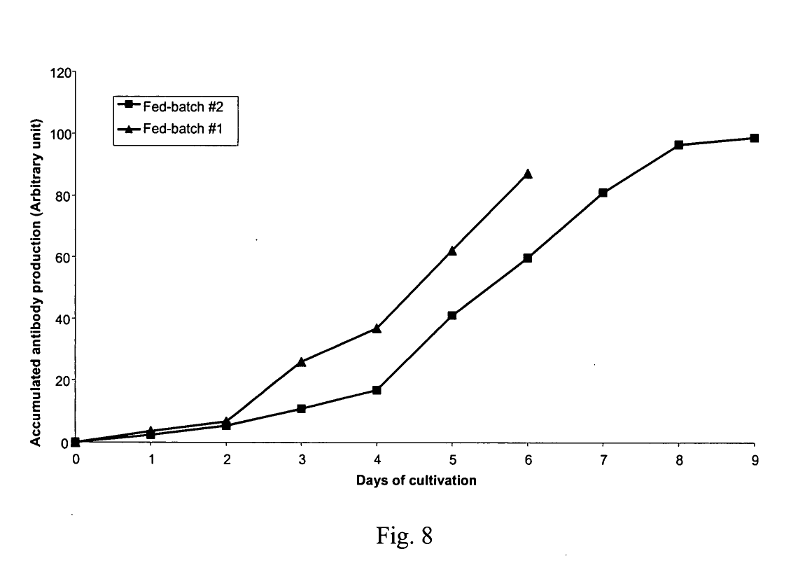

Two fed-batch cultivations, fed-batch #1 and fed-batch #2, were performed with an antibody producing CHO cell line in 3 L bioreactors using a basal medium based on DMEM/F12 medium enriched in vitamins, metals, biosynthesis precursors and pyruvate, and supplemented with a combination of peptones G and E (50/50%/%) at a total concentration of 5 g/L. The cultivations were fed continuously from day 2 with glucose, glutamine, amino acid cocktail and concentrated feed medium consisting of the basal medium enriched in vitamins, metals and biosynthesis precursors. To the fed-batch #2, the feed also included a combination of peptones G and E (50/50%/%) fed continuously at total concentration of 0.6 g/L/day. Both cultures were terminated when the cell viability was between 70-80%. FIG. 7 shows an improvement of the cell viability and hence the process longevity when a combination of cotton seed and pea protein hydrolysates was fed to the culture. FIG. 8 shows an increase of antibody production when a combination of cotton seed and pea protein hydrolysates was fed to the culture.
peptone E=cotton seed protein hydrolysate
peptone G=pea protein hydrolysate

### Example 5

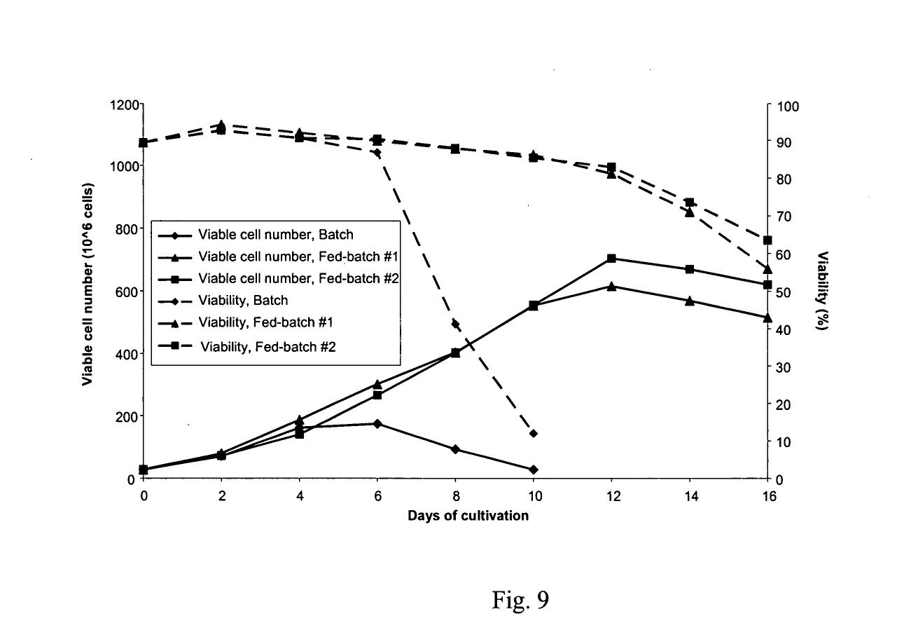

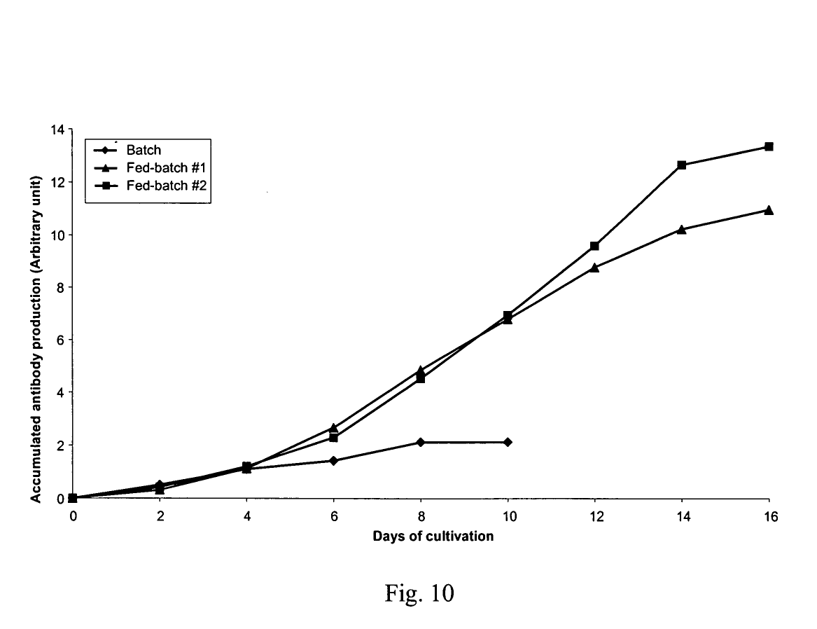

Two fed-batch cultivations, fed-batch #1 and fed-batch #2, were performed with an antibody producing CHO cell line in spinners using a basal medium based on DMEM/F12 medium enriched with disclosed additives including surfactant, trace elements, amino acids, vitamins, growth factors, and supplemented with a combination of peptones G and E (50/50%/%) at a total concentration of 5 g/L. The cultivations were fed every other day with glucose, glutamine, and concentrated basal medium. To the fed-batch #2, the feed also included a combination of peptones G and E (50/50%/%) fed every other day at total concentration of 1.2 g/L. A batch cultivation in the same disclosed basal medium supplemented with a combination of peptones G and E (50/50%/%) at a total concentration of 5 g/L was performed as a reference. FIG. 9 shows a significant increase of viable cell number and a significant improvement of cell viability in the fed-batch cultures. Feeding cotton seed and pea protein hydrolysates further increased the viable cell number and cell viability. FIG. 10 shows a significant increase of antibody production in the fed-batch cultures. Feeding cotton seed and pea protein hydrolysates further increased the antibody production.
peptone E=cotton seed protein hydrolysate
peptone G=pea protein hydrolysate

### Example 6

This example shows that the beneficial effects of peptone feeding cannot be reproduced by supplementation of amino acids. Over-feeding amino acids may be toxic to the cells. The example also shows that addition of the peptone or combination of peptones can partially neutralize the toxic effect from over-feeding of the amino acids during a fed-batch process, resulting in improvements of viable cell number, cell viability, process longevity, and productivity. Six fed-batch cultivations, fed-batch #1, fed-batch #2, fed-batch #3, fed-batch #4, fed-batch #5, fed-batch #6, were performed in duplicates with an antibody producing CHO cell line in 50 ml filtered tubes using a basal medium based on DMEM/F12 medium enriched in vitamins, metals, biosynthesis precursors and pyruvate, and supplemented with a combination of peptones G and E (50/50%/%) at a total concentration of 5 g/L (Table 1).

TABLE 1

| | Feed | | | |
|---|---|---|---|---|
| | Glucose, | | Peptones G | Amio acid |
| Exp. ID | glutamine | Feed medium | and E | cocktail |
| Batch | No | No | No | No |
| Fed-batch | Yes | Yes | 0.8 g/L | No |
| #1 | on days 2, 4, | on days 3, 6, 9 | on days 2, 4; | |
| | 6, 8 | | 0.4 g/L | |
| | | | on days 6, 8 | |
| Fed-batch | Yes | Yes | 1.6 g/L | No |
| #2 | as fed-batch | as fed-batch #1 | on days 2, 4; | |
| | #1 | | 0.8 g/L | |
| | | | on days 6, 8 | |
| Fed-batch | Yes | Yes | No | 0.4 ml |
| #3 | as fed-batch | as fed-batch #1 | | on days 2, 4, 6; |
| | #1 | | | 0.2 ml |
| | | | | on day 8 |
| Fed-batch | Yes | Yes | No | 1.6 ml |
| | as fed-batch | as fed-batch #1 | | on days 2, 4, 6; |
| | #1 | | | 0.8 ml on day 8 |
| Fed-batch | Yes | Yes | 0.8 g/L | 0.4 ml on |
| #5 | as fed-batch | as fed-batch #1 | on days 2, 4; | on days 2, 4, 6; |
| | #1 | | 0.4 g/L | 0.2 ml |
| | | | on days 6, 8 | on day 8 |
| Fed-batch | Yes | Yes | 1.6 g/L | 0.4 ml |
| #6 | as fed-batch | as fed-batch #1 | on days 2, 4; | on days 2, 4, 6; |
| | #1 | | 0.8 g/L | 0.2 ml |
| | | | on days 6, 8 | on day 8 |

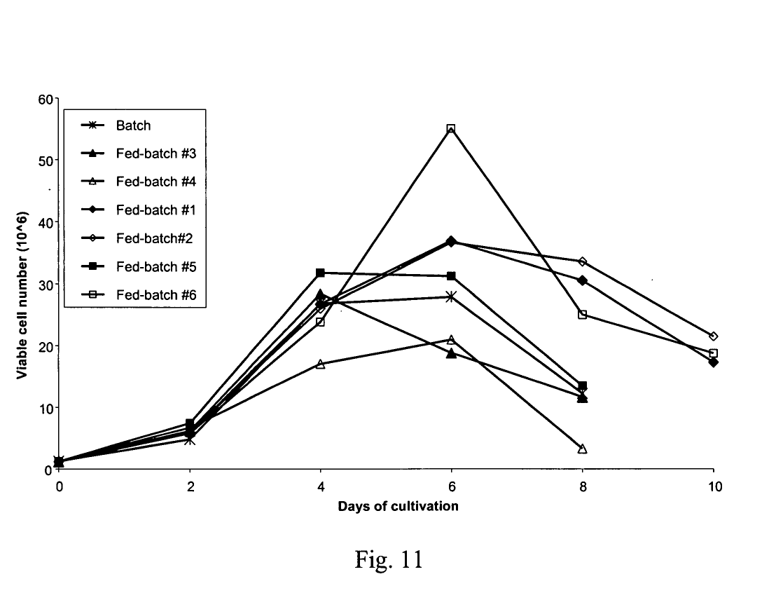

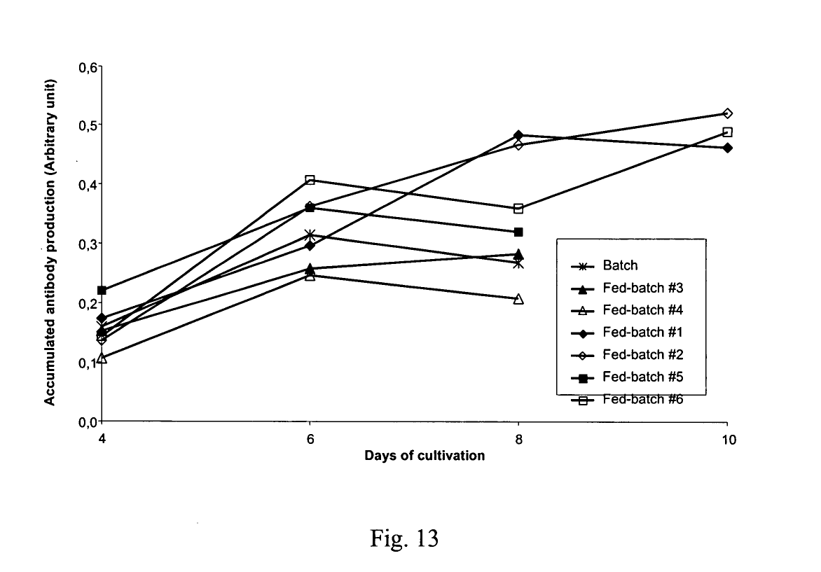

The fed-batch #1 was fed with glucose, glutamine, concentrated feed medium consisting of the basal medium enriched in vitamins, metals and biosynthesis precursors. A combination of peptones G and E (50/50%/%) was also added to the fed-batch #1 at a total dose of 0.8 g/L on days 2 and 4, and 0.4 g/L on days 6 and 8. The fed-batch #2 was fed exactly as to the fed-batch #2, but the dose of peptone feeding was doubled. The fed-batch #3 was fed exactly as to the fed-batch #1, but the peptone feeding was replaced by feeding with the amino acid cocktail at a total dose of 0.4 ml on days 2, 4, and 6, and 0.2 ml on day 8. The fed-batch #4 was fed exactly as to the fed-batch #3, but the dose of the amino acid cocktail feeding was increased 4 folds. The fed-batch #5 was fed exactly as to the fed-batch #3, plus a peptone feeding with the same dose as to the fed-batch #1. The fed-batch #6 was fed exactly as to the fed-batch #3, plus a peptone feeding with the same dose as to the fed-batch #2. A batch cultivation in the same basal medium supplemented with a combination of peptones G and E (50/50%/%) at a total concentration of 5 g/L was performed as a reference. The average values from the duplicate cultures were presented in the FIGS. 11-13. It was found that viable cell number, cell viability, and antibody production were significantly increased in the fed-batch cultures #1 and #2, as compared to the batch culture. Lower viable cell number, cell viability, and antibody production were obtained when the peptone feeding was replaced with feeding with the amino acid cocktail (fed-batch #3 and #4). Feeding higher dose of amino acid cocktail in the fed-batch #4 was more toxic to the cells. Surprisingly, when the peptones were fed together with the amino acid cocktail in the fed-batch #5 and #6, the viable cell number, cell viability, and antibody production were improved as compared to the fed-batch #3. The addition of higher dose of peptones in the fed-batch #6 could neutralize the toxic effect of the amino acid cocktail feeding to a great extend, resulting in comparable viable cell number and antibody production as in the fed-batch #1 and #2.
peptone E=cotton seed protein hydrolysate
peptone G=pea protein hydrolysate

### Example 7

This example shows that addition of the peptone or combination of peptones can partially neutralize the toxic effect from over-feeding of the amino acids during a fed-batch process, resulting in improvements of viable cell number, cell viability, process longevity, and productivity. Four fed-batch cultivations, fed-batch #1, #2, #3, and #4, were performed with an antibody producing CHO cell line in spinners using a basal medium based on DMEM/F12 medium enriched in vitamins, metals, biosynthesis precursors and pyruvate, and supplemented with a combination of peptones G and E (50/50%/%) at a total concentration of 5 g/L. All the four fed-batch cultures were fed with glucose, glutamine, and concentrated feed medium consisting of the basal medium enriched in vitamins, metals and biosynthesis precursors (Table 2).

TABLE 2

| | Feed | | | |
|---|---|---|---|---|
| | Glucose, | | | Amio acid |
| Exp. ID | glutamine | Feed medium | Peptones G and E | cocktail |
| Batch | No | No | No | No |
| Fed-batch #1 | Yes | Yes | No | No |
| | on days 2, 4, 6, 8 | on days 3, 6, 9 | | |
| Fed-batch #2 | Yes | Yes | 1.2 g/L | No |
| | as fed-batch #1 | as fed-batch #1 | on days 2, 4, 6, 8 | |
| Fed-batch #3 | Yes | Yes | No | Yes |
| | as fed-batch #1 | as fed-batch #1 | | on days 2, 4, 6, 8 |
| Fed-batch #4 | Yes | Yes | 1.2 g/L | Yes |
| | as fed-batch #1 | as fed-batch #1 | on days 2, 4, 6, 8 | as fed-batch #3 |
| | | | as fed-batch #2 | |

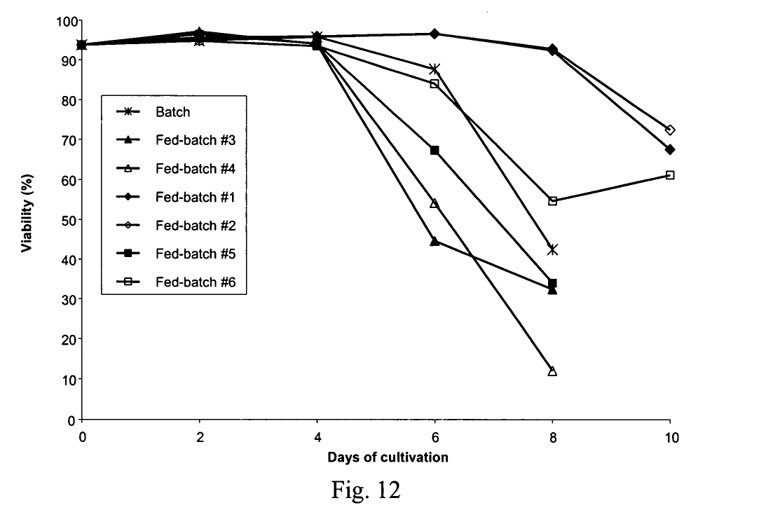

To the fed-batch #2, the feed also included a combination of peptones G and E (50/50%/%) at a total dose of 1.2 g/L every other day (feed medium+peptones). To the fed-batch #3, the feed also included a cocktail of amino acid (feed medium+amino acid cocktail). To the fed-batch #4, the feed also included a combination of peptones G and E (50/50%/%) as well as a cocktail of amino acids with the same doses as fed to the fed-batch #2 and #3, respectively (feed medium+peptones+amino acid cocktail). A batch cultivation in the same basal medium supplemented with a combination of peptones G and E (50/50%/%) at a total concentration of 5 g/L was performed as a reference. FIG. 11 shows an increase of viable cell number and an improvement of cell viability when a combination of cotton seed and pea protein hydrolysates was fed to the culture (#2 vs. #1). The viable cell number and cell viability decreased when the amino acid cocktail was fed to the culture (#3 vs. #1), indicating a toxic effect from the amino acid feeding. The viable cell number and cell viability were improved when both the amino acid cocktail and the peptones were fed to the culture (#4 vs. #3), indicating that addition of the peptones partially neutralized the toxic effect from over-feeding of the amino acids. As shown in FIG. 12, the fed-batch #2 gave the highest antibody production, followed by the fed-batch #1, the fed-batch #4, and then the fed-batch #3. As expected, the batch culture had the poorest cell growth and the lowest antibody production.
peptone E=cotton seed protein hydrolysate
peptone G=pea protein hydrolysate

### Example 8

This example demonstrates an increase in the viable cell density and cell viability when feeding peptones G and E, and not just only when the peptones G and E are present in the basal medium, i.e. during the whole cultivation. Three fed-batch cultivation runs, runs #1, #2 and #3, are performed using a basal medium based on DMEM/F12 medium enriched with a list of disclosed additives including surfactant, trace elements, amino acids, vitamins, and growth factors, and with or without the supplementation with a peptone or a combination of peptones. The cultivation is fed with several components, i.e. glucose, glutamine, amino acid cocktail and concentrated feed medium consisting of the basal medium enriched in disclosed additives including vitamins, metals and biosynthesis precursors. When the viable cell density and the cell viability have begun to decrease, the amino acid cocktail is replaced in run #3 by feeding a cocktail including peptones G and E. Peptone D and peptone G are added to the basal medium. As control, the fed-batch cultivation run # 1 is performed in exactly the same conditions as run #3 except that the amino acid cocktail is not replaced by a peptone feeding but continued in the same way. Fed-batch cultivation run #2 is performed according to the same conditions as run #1 except that the basal medium is supplemented with peptone E and peptone G instead of peptone D and peptone G. In run #3, peptone G and E feeding is applied after the viable cell density begun to decrease, which result in a cell increase and viability increase, while in run #1, the viable cell density and cell viability will continue to decrease. Run #2 has a basal medium including the precise peptone combination G and E.
peptone D=soy protein hydrolysate
peptone E=cotton seed protein hydrolysate
peptone G=pea protein hydrolysate

### OTHER EMBODIMENTS

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A process for cultivating animal cells producing complex proteins, wherein one plant-derived peptone or a combination of plant-derived peptones is fed to a cell culture.

2. The process of claim 1, wherein a basal medium is used for cell inoculation and a feed medium is fed to the cell culture.

3. The process of claim 1, wherein the process is a fed batch process.

4. The process of claim 1, wherein the process is a fed perfusion process.

5. The process of claim 1, wherein the cultivated cells are secreting proteins.

6. The process of claim 5, wherein the secreted proteins are antibodies.

7. The process of claim 1, wherein feeding of the peptone or combination of peptones is started before the cell culture reaches stationary phase.

8. The process of claim 1, wherein feeding of the peptone or combination of peptones is started when the cell culture has reached stationary phase.

9. The process of claim 1, wherein feeding of the peptone or combination of peptones is started at any time between cell inoculation and before cell viability decreases below viability at cell inoculation.

10. The process of claim 9, wherein feeding of the peptone or combination of peptones is started at any time between cell inoculation and three days before cell viability decreases below viability at cell inoculation.

11. The process of claim 10, wherein feeding of the peptone or combination of peptones is started at any time between cell inoculation and three days before cell viability decreases below viability at cell inoculation.

12. The process of claim 1, wherein feeding of the peptone or combination of peptones is started at any time between cell inoculation and before cell viability begins to decrease.

13. The process of claim 1, wherein the peptone or combination of peptones is fed when the viable cell density and /or the cell viability has begun to decrease.

14. The process of claim 1, wherein one peptone is fed to the cell culture.

15. The process of claim 14, wherein the peptone is derived from Fabaceae vicieae protein.

16. The process of claim 1, wherein a combination of peptones is fed to the cell culture.

17. The process of claim 16, wherein the combination of peptones comprises a peptone produced by enzymatic digest and which is derived from protein of the Fabaceae family vicieae tribe.

18. The process of claim 17, wherein the peptone is derived from Pisum sativum (pea).

19. The process of claim 18, wherein the combination of peptones further comprises peptones derived from Fabaceae glycine max protein (soy), or Malvaceae seed protein, or both.

20. The process of claim 19, wherein the peptone derived from the seed protein of a Malvaceae is Malvaceae gossypium (cotton seed protein).

21. The process of claim 1, wherein the total dose for the process corresponds to a total concentration of peptone or combination of peptones fed to the cell culture from and including 0.01 gram per litre of cultivation volume at inoculation to and including 15 gram per litre of cultivation volume at inoculation.

22. The process of claim 21, wherein the total dose for the process corresponds to a total concentration of peptone or combination of peptones fed to the cell culture from and including 0.01 gram per litre of cultivation volume at inoculation to and including 11 gram per litre of cultivation volume at inoculation.

23. The process of claim 22, wherein the total dose for the process corresponds to a total concentration of peptone or combination of peptones fed to the cell culture from and including 0.01 gram per litre of cultivation volume at inoculation to and including 5 gram per litre of cultivation volume at inoculation.

24. The process of claim 1, wherein the animal cells are mammalian cells.

25. The process of claim 24, wherein the mammalian cells are human cells or rodent cells.

26. The process of claim 25, wherein the rodent cells are hamster cells.

27. The process of claim 26, wherein the hamster cells are Chinese Hamster Ovary cells.

28. The process of claim 1, wherein the feed medium is added continuously to the cell culture.

29. The process of claim 1, wherein the feed medium is added intermittently to the cell culture.

30. The process of claim 1, wherein the feed medium is added boost-wise to the cell culture.

31. The process of claim 1, wherein the basal medium contains peptones.

32. A method for reducing the toxic effect of over-feeding amino acids during a fed-batch process for cultivating animal cells producing complex proteins, by feeding one plant-derived peptone or a combination of plant-derived peptones to the cell culture according to the process of claim 1.
